(19) United States
(12) Patent Application Publication
(10) Pub. No.: US 2007/0275394 A1
(43) Pub. Date: Nov. 29, 2007

(54) Nucleic acid nanostructure and method of manufacturing the same

(76) Inventors: Dong Ho Shin, Daejeon-city (KR); Moon Youn Jung, Daejeon-city (KR); Se Ho Park, Daejeon-city (KR); Hyeon Bong Pyo, Daejeon-city (KR); Chang Auck Choi, Daejeon-city (KR)

Correspondence Address: LADAS & PARRY LLP, 224 SOUTH MICHIGAN AVENUE, SUITE 1600, CHICAGO, IL, 60604, US

(21) Appl. No.: 11/634,437

(22) Filed: Dec. 6, 2006

(30) Foreign Application Priority Data
Dec. 8, 2005 (KR) 10-2005-0119524
May 24, 2006 (KR) 10-2006-0046521

(51) Int. Cl. C12Q 1/68; C12M 3/00

(52) U.S. Cl. 435/6; 435/287.2; 977/924; 427/2.11

(57) ABSTRACT

Provided are a nucleic acid nanostructure including: a substrate; a nucleic acid quadruplex immobilized on the substrate to be vertical with respect to the substrate; a metal ion present in a unit lattice of the nucleic acid quadruplex, the unit lattice being made up of eight nucleobases; and a nanoparticle bound to an end of the nucleic acid quadruplex, and a method of manufacturing the same. According to the method, a nucleic acid nanostructure having an array of nanoparticles can be manufactured. The nucleic acid nanostructure can be applied as a sensor nanostructure for sensors such as gas sensors, chemical sensors, and biosensors. In particular, a nucleic acid nanostructure in which metal nanoparticles, e.g., gold or silver, are introduced can be useful as a device having local surface plasmon characteristics.

### CROSS-REFERENCE TO RELATED PATENT APPLICATION

This application claims priority from Korean Patent Application No. 10-2005-0119524 filed on Dec. 08, 2005 and No. 10-2006-0046521 filed on May 24, 2006, in the Korean Intellectual Property Office, the disclosures of which are incorporated herein in their entireties by reference.

1. Field of the Invention

The present invention relates to a nucleic acid nanostructure and a method of manufacturing the same. More particularly, the present invention relates to a nucleic acid nanostructure using a nucleic acid quadruplex structure and a method of manufacturing the same.

2. Description of the Related Art

Nucleic acids, such as DNAs, are known to have nanostructures in specific conditions. Based on this finding, research about development of nanostructures or nanodevices that can be used in sensors for detecting gases, chemical substances, or biomolecules has been actively conducted.

Such research has been focused on the employment of single strands or double-helix structure of DNA. Most research is conducted based on hybridization of DNA laid on a surface of a substrate with its complementary sequence. For this, a self-assembly process is used in which template DNAs are synthesized and dissolved in an appropriate solution, the DNA-containing solution is coated on a substrate, and the DNAs are self-assembled on the substrate in appropriate conditions.

However, according to the above process, local formation of desired nanostructures is enabled, but it is difficult to form nanostructures over a broad area and the reproducibility of the nanostructures is also poor.

Generally, DNAs in vivo are known to have a double-helix structure. However, the existence of DNAs having a different structure (e.g., triplex or quadruplex) from a double-helix structure in specific conditions or sites has been discovered. As the newly discovered DNA structures are known to have a physiological or pathological importance, they are of much interest to researchers.

Through various experiments, it is found that a different structure from a double-helix structure can be formed by repeated arrangement of single nucleotide molecules. In particular, guanine (G)-rich sequences are found to form a hydrogen-bond pairing of four guanines, which is structurally different from a guanine-cytosine base pairing. Such a unit structure is called “G-quadruplex” or “G-quartet”.

FIG. 1 is a diagram illustrating a G-quadruplex structure made up of a G-tetrad. Referring to FIG. 1, when a metal ion is centered in the G-tetrad, the G-quadruplex structure is more stabilized. Such structural characteristics are also found in RNAs, PNAs, LNAs, or derivatives of other nucleobases. Various quadruplex structures are known.

However, nucleic acid quadruplex-based nanostructures or nanodevices that can be used in sensors for detecting gases, chemical substances, or biomolecules have not yet been reported.

### SUMMARY OF THE INVENTION

The present invention provides a nucleic acid nanostructure including high-density nanoparticles over a broad area.

The present invention also provides a method of manufacturing a nucleic acid nanostructure including high-density nanoparticles over a broad area with high reproducibility.

According to an aspect of the present invention, there is provided a nucleic acid nanostructure including: a substrate; a nucleic acid quadruplex immobilized on the substrate to be vertical with respect to the substrate; a metal ion present in a unit lattice of the nucleic acid quadruplex, the unit lattice being made up of eight nucleobases; and a nanoparticle bound to an end of the nucleic acid quadruplex.

The substrate may be selected from the group consisting of a metal substrate, a glass substrate, a semiconductor wafer, a quartz substrate, and a plastic substrate.

The nucleic acid may be selected from the group consisting of DNA, RNA, PNA, LNA, and a hybrid thereof.

The nucleic acid quadruplex may be composed of four nucleic acid strands which are arranged in a parallel or antiparallel orientation.

The nucleic acid quadruplex may be composed of four nucleic acid strands which are arranged in parallel with each other in a 5′ to 3′ direction from the substrate.

Each of the four nucleic acid strands of the nucleic acid quadruplex may include a guanine-rich sequence.

Each of the four nucleic acid strands of the nucleic acid quadruplex may include a sequence selected from the group consisting of sequences as set forth in SEQ ID NOS: 1 through 3.

The metal ion may be selected from the group consisting of Na⁺, K⁺, Mg²⁺, Ca²⁺, Mn²⁺, Ni²⁺, Cd²⁺, Co²⁺, and Zn²⁺.

The nanoparticle may be at least one selected from the group consisting of Au, Ag, ZnS, CdS, CdSe, SiO₂, SnO₂, TiO₂, GaAs, and InP.

According to another aspect of the present invention, there is provided a method of manufacturing a nucleic acid nanostructure, the method including: introducing a nucleic acid capable of forming a quadruplex onto a substrate; forming a nucleic acid quadruplex from the introduced nucleic acid; and binding a nanoparticle to an end of the nucleic acid quadruplex.

In the introduction of the nucleic acid, a functional group may be bound to an end of the nucleic acid capable of forming the quadruplex, and the functional group-containing nucleic acid may be immobilized on the substrate.

In introduction of the nucleic acid, the nucleic acid capable of forming the quadruplex may be in-situ grown on the substrate.

In the formation of the nucleic acid quadruplex, a metal ion may be supplied to the introduced nucleic acid.

In the binding of the nanoparticle, the nanoparticle may be supplied to the nucleic acid quadruplex.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating a guanine (G)-quadruplex structure made up of a G-tetrad;

FIG. 2 is a schematic perspective view illustrating a nucleic acid nanostructure according to an embodiment of the present invention;

FIG. 3 is a schematic diagram illustrating various nucleic acid quadruplex structures that can be made using 1-4 nucleic acid strands;

FIG. 4A is a schematic diagram illustrating an embodiment of a nucleic acid introduction process in a method of manufacturing a nucleic acid nanostructure according to the present invention;

FIG. 4B is a schematic diagram illustrating another embodiment of a nucleic acid introduction process in a method of manufacturing a nucleic acid nanostructure according to the present invention;

FIG. 4C is a schematic diagram illustrating an embodiment of a nucleic acid quadruplex formation process in a method of manufacturing a nucleic acid nanostructure according to the present invention;

FIG. 4D is a schematic diagram illustrating a nucleic acid quadruplex manufactured using the method illustrated in FIG. 4C;

FIG. 4E is a schematic diagram illustrating an embodiment of a nanoparticle binding process in a method of manufacturing a nucleic acid nanostructure according to the present invention, and a nucleic acid nanostructure manufactured using the method; and

FIG. 5 is a diagram illustrating an array of unit nanostructures of a nucleic acid nanostructure manufactured using the method illustrated in FIG. 4E.

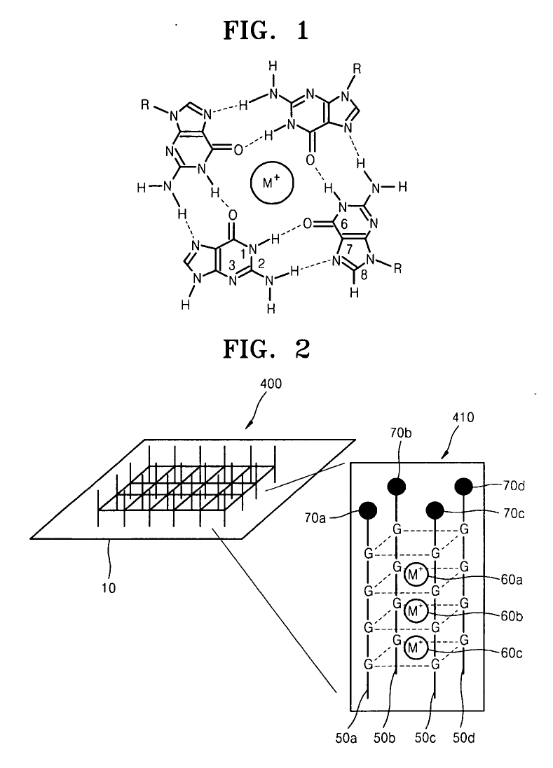

### DETAILED DESCRIPTION OF THE INVENTION

The present invention will now be described more fully with reference to the accompanying drawings, in which exemplary embodiments of the invention are shown.

The present invention provides a nucleic acid nanostructure that can be used in sensors for detecting various substances. More particularly, the present invention provides a nucleic acid nanostructure including: a substrate; a nucleic acid quadruplex immobilized on the substrate to be vertical with respect to the substrate; a metal ion present in a unit lattice of the nucleic acid quadruplex, the unit lattice being made up of eight nucleobases; and a nanoparticle bound to an end of the nucleic acid quadruplex.

FIG. 2 is a schematic perspective view illustrating a nucleic acid nanostructure according to an embodiment of the present invention.

Referring to FIG. 2, a nucleic acid nanostructure 400 according to an embodiment of the present invention includes a substrate 10; a nucleic acid quadruplex composed of four nucleic acid strands 50a, 50b, 50c, and 50d which are immobilized on the substrate 10 to be vertical with respect to the substrate 10; metal ions 60a, 60b, and 60c present in unit lattices of the nucleic acid quadruplex, each unit lattice being made up of eight nucleobases; and nanoparticles 70a, 70b, 70c, and 70d bound to ends of the four nucleic acid strands 50a, 50b, 50c, and 50d of the nucleic acid quadruplex.

In the nucleic acid nanostructure of the present invention, the substrate is not particularly limited. For example, the substrate may be selected from the group consisting of a metal substrate, a glass substrate, a semiconductor wafer, a quartz substrate, and a plastic substrate.

The type of the nucleic acid is not particularly limited. For example, the nucleic acid may be selected from the group consisting of DNA, RNA, PNA, LNA, and a hybrid thereof.

The nucleic acid quadruplex may be derived from all nucleic acid combinations locally forming a quadruplex. That is, the nucleic acid quadruplex may be variously structured using 1-4 nucleic acid strands.

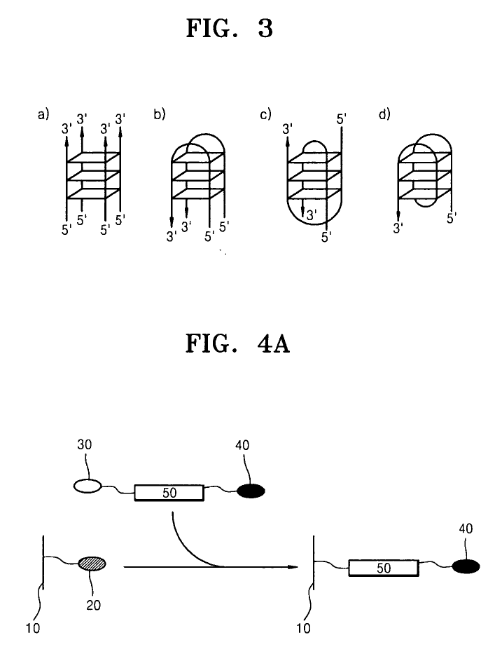

FIG. 3 is a schematic diagram illustrating various nucleic acid quadruplex structures that can be made using 1-4 nucleic acid strands. Referring to FIG. 3, a nucleic acid quadruplex may be composed of four strands (see a) of FIG. 3), two strands (see b) and c) of FIG. 3), and a single strand (see d) of FIG. 3). In addition, various quadruplexes may be formed according to the nucleotide sequence of each strand.

Preferably, the nucleic acid quadruplex may be composed of four strands which are arranged in a parallel or antiparallel orientation. That is, the four strands of the nucleic acid quadruplex may be arranged, in parallel with each other, or one or two strands of the nucleic acid quadruplex may be arranged in an antiparallel orientation with respect to the other strands.

As used herein, the term “parallel” means that two nucleic acid strands are arranged in a 5′ to 3′ direction, and the term “antiparallel” means that one of two nucleic strands is arranged in a 5′ to 3′ direction and the other strand is arranged in a 3′ to 5′ direction.

More preferably, the nucleic acid quadruplex may be composed of four strands which are arranged in parallel with each other in the 5′ to 3′ direction from the substrate.

The four strands of the nucleic acid quadruplex may include any nucleic acid sequences capable of binding with each other to form a nucleic acid quadruplex structure. For example, each of the four strands of the nucleic acid quadruplex may include a guanine-rich sequence. Preferably, each of the four strands of the nucleic acid quadruplex may include a nucleic acid sequence selected from the group consisting of nucleic acid sequences as set forth in SEQ ID NOS: 1-3. In the sequence listing attached to the specification, it should be understood by one of ordinary skill in the art that thymine (T) is replaced by uracil (U) in RNA.

For a detailed description of a nucleic acid quadruplex, reference can be made, for example, to U.S. Pat. Nos. 6,017,709, 6,900,300, and 6,656,692.

In the nucleic acid nanostructure of the present invention, the metal ion is not particularly limited. For example, the metal ion may be selected from the group consisting of Na⁺, K⁺, Mg²⁺, Ca²⁺, Mn²⁺, Ni²⁺, Cd²⁺, Co²⁺, and Zn²⁺.

The nanoparticle may be at least one selected from the group consisting of Au, Ag, ZnS, CdS, CdSe, SiO₂, SnO₂, TiO₂, GaAs, and InP. In particular, a nucleic acid nanostructure including a metal nanoparticle (e.g., Au or Ag) can be useful as a device with local surface plasmon characteristics.

The present invention also provides a method of manufacturing a nucleic acid nanostructure.

The method of manufacturing the nucleic acid nanostructure according to the present invention includes: introducing a nucleic acid capable of forming a quadruplex onto a substrate; forming a nucleic acid quadruplex from the introduced nucleic acid; and binding nanoparticles to an end of the nucleic acid quadruplex.

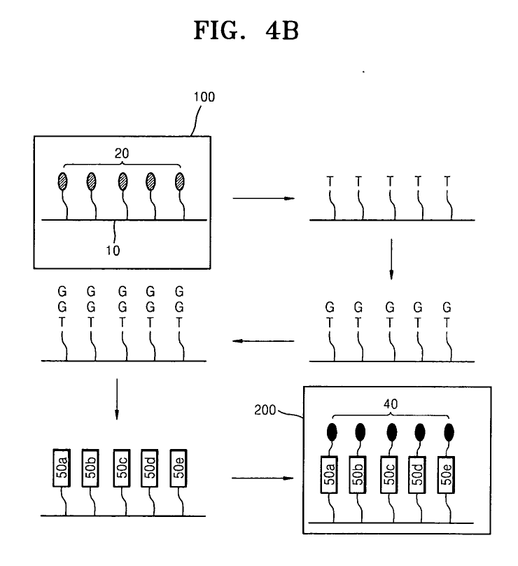

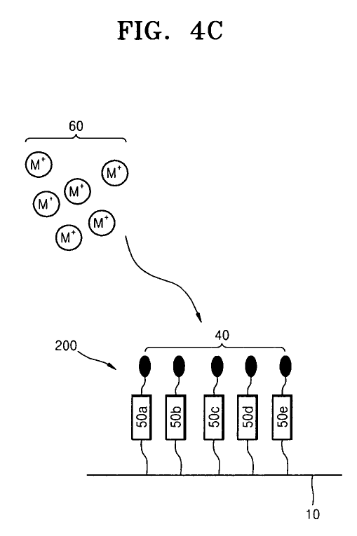

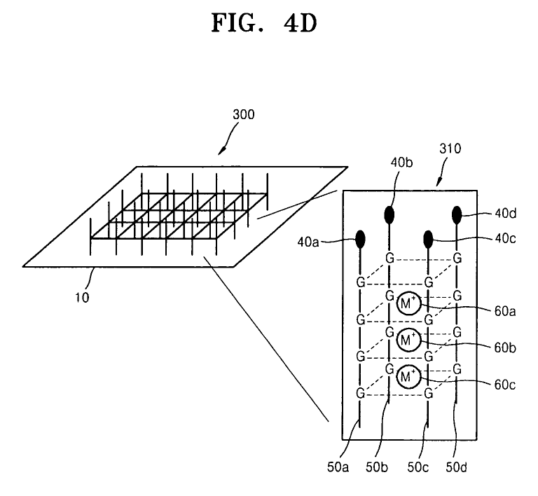

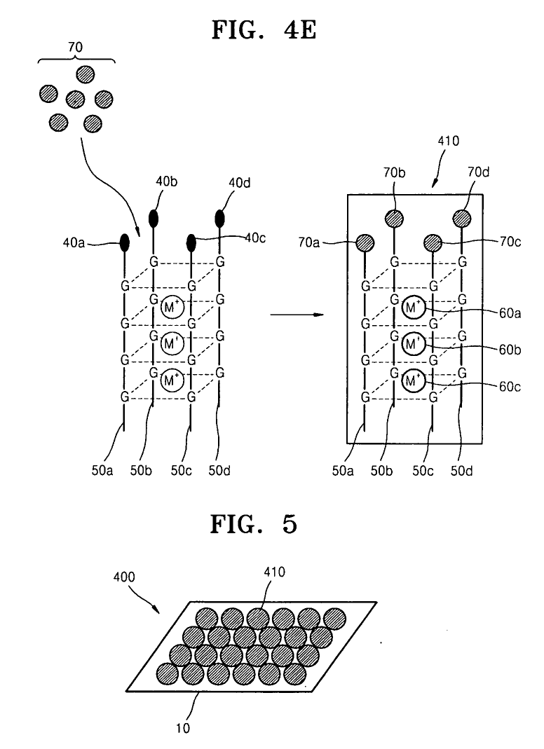

FIGS. 4A through 4E are schematic diagrams illustrating a method of manufacturing a nucleic acid nanostructure according to an embodiment of the present invention. Hereinafter, a method of manufacturing a nucleic acid nanostructure according to an embodiment of the present invention will be described in more detail with reference to FIGS. 4A through 4E.

In the method of manufacturing the nucleic acid nanostructure according to the present invention, detailed descriptions of a substrate, a nucleic acid, a nucleic acid quadruplex, metal ions, and nanoparticles are as described above.

<Nucleic Acid Introduction>

In order to manufacture a nucleic acid nanostructure, first, a nucleic acid capable of forming a quadruplex is introduced onto a substrate. The introduction of the nucleic acid onto the substrate can be performed using a commonly known method for immobilizing a nucleic acid on a substrate.

For example, the introduction of the nucleic acid onto the substrate can be performed by binding a functional group to an end of a nucleic acid capable of forming a quadruplex and immobilizing the functional group-containing nucleic acid onto a substrate. FIG. 4A is a schematic diagram illustrating an embodiment of a nucleic acid introduction process in a method of manufacturing a nucleic acid nanostructure according to the present invention.

Referring to FIG. 4A, a nucleic acid 50 capable of forming a quadruplex is synthesized. Then, a functional group 30 capable of binding with a substrate 10 having a functional group 20 on a surface thereof is attached to an end of the nucleic acid 50, and a functional group 40 capable of binding with nanoparticles (not shown) is attached to the other end of the nucleic acid 50. Then, the nucleic acid 50 thus modified is supplied and immobilized onto the substrate 10.

Here, the functional groups 20, 30, and 40 may be selected from functional groups capable of realizing covalent bonds or antigen-antibody interactions. For example, the functional group 20 may be a functional group which can be introduced onto a surface using a conventional surface modification process, e.g., a carboxyl group, a thiol group, a hydroxyl group, a silane group, an amine group, or an epoxy group.

With respect to spotting of a previously prepared nucleic acid onto a predetermined region of a substrate, reference can be made, for example, to U.S. Pat. No. 5,807,522 and WO 98/18961.

Alternatively, the introduction of the nucleic acid onto the substrate can be performed by growing a nucleic acid capable of forming a quadruplex on a substrate using an in-situ process. FIG. 4B is a schematic diagram illustrating another embodiment of a nucleic acid introduction process in a method of manufacturing a nucleic acid nanostructure according to the present invention.

Referring to FIG. 4B, thymine (T) is attached onto a surface of a substrate 10 having a functional group 20, and predetermined nucleobases are then attached thereto by successive layering to prepare nucleic acids 50a, 50b, 50c, 50d, and 50e capable of forming quadruplexes. Then, a functional group 40 capable of binding with nanoparticles (not shown) is attached to ends of the nucleic acids 50a, 50b, 50c, 50d, and 50e to thereby manufacture a substrate surface 200 on which the nucleic acids 50a, 50b, 50c, 50d, and 50e are introduced.

With respect to a method of synthesizing single-stranded DNAs on predetermined regions of a substrate, reference can be made, for example, to U.S. Pat. Nos. 5,445,934, 5,744,305, and 5,700,637.

<Quadruplex Formation >

Next, a nucleic acid quadruplex is formed from the introduced nucleic acid.

The formation of the nucleic acid quadruplex can be performed by supplying a metal ion to the nucleic acid immobilized as described above. The metal ion is not particularly limited, and illustrative examples thereof are as described above.

The formation of the nucleic acid quadruplex can be performed in a common medium known to be suitable to conserve nucleotides.

FIG. 4C is a schematic diagram illustrating an embodiment of a nucleic acid quadruplex formation process in a method of manufacturing a nucleic acid nanostructure according to the present invention. Referring to FIG. 4C, together with FIG. 4B, metal ions (M⁺) 60 are supplied to the substrate surface 200 prepared above.

FIG. 4D is a schematic diagram illustrating a nucleic acid quadruplex manufactured using the method illustrated in FIG. 4C. Referring to FIG. 4D, guanine (G) tetrads are hydrogen-bonded in the presence of metal ions 60a, 60b, and 60c to form a quadruplex unit structure 310 composed of four strands 50a, 50b, 50c, and 50d. The quadruplex unit structure 310 shows a structure of unit lattices, and each unit lattice is structured such that one metal ion is trapped in a hexahedron composed of eight guanines. The unit lattices are kinds of crystals and form a quadruplex crystal structure 300 having an interstitial interval of 1˜2 nm. Once formed, the quadruplex crystal structure 300 is maintained in a very stable state at room temperature.

<Nanoparticle Binding>

Next, nanoparticles are bound to an end of the above-prepared nucleic acid quadruplex.

The binding of the nanoparticles to the nucleic acid quadruplex can be performed by supplying the nanopaticles to the nucleic acid quadruplex. The nanoparticles are not particularly limited, and illustrative examples thereof are as described above.

The binding of the nanoparticles to the nucleic acid quadruplex can be performed in a common medium known to be suitable to conserve nucleotides.

FIG. 4E is a schematic diagram illustrating an embodiment of a nanoparticle binding process in a method of manufacturing a nucleic acid nanostructure according to the present invention, and a nucleic acid nanostructure manufactured using the method.

Referring to FIG. 4E, together with FIG. 4D, nanoparticles 70 are supplied to the quadruplex unit structure 310 of the quadruplex crystal structure 300. Functional groups 40a, 40b, 40c, and 40d capable of covalently binding with the nanoparticles 70 are present on ends of nucleic acid strands 50a, 50b, 50c, and 50d constituting the quadruplex unit structure 310 of the quadruplex crystal structure 300. As a result, the nanoparticles 70 are broadly distributed in high density in a predetermined concentration over the quadruplex crystal structure 300 in such a manner that nanoparticles 70a, 70b, 70c, and 70d are covalently bound to the functional groups 40a, 40b, 40c, and 40d.

FIG. 5 is a diagram illustrating an array of unit structures of a nucleic acid nanostructure manufactured using the method illustrated in FIG. 4E. Referring to FIG. 5, a nucleic acid nanostructure 400 is structured such that a plurality of unit structures 410 are arranged on a substrate 10.

As described above, according to a method of manufacturing a nucleic acid nanostructure of the present invention, a nucleic acid nanostructure having an array of nanoparticles can be manufactured. A nucleic acid nanostructure according to the present invention can be applied as a sensor nanostructure for sensors such as gas sensors, chemical sensors, and biosensors. In particular, a nucleic acid nanostructure in which metal nanoparticles, e.g., gold or silver, are introduced can be useful as a device having local surface plasmon characteristics.

While the present invention has been particularly shown and described with reference to exemplary embodiments thereof, it will be understood by those of ordinary skill in the art that various changes in form and details may be made therein without departing from the spirit of the present invention. Thus, the embodiments are to be considered in all respects as illustrative and not restrictive. The scope of the present invention is defined by the following claims, not by the above detailed description. It should be understood that all equivalents of the embodiments are within the scope of the present invention.

What is claimed is:

1. A nucleic acid nanostructure comprising:
a substrate;
a nucleic acid quadruplex immobilized on the substrate to be vertical with respect to the substrate;
a metal ion present in a unit lattice of the nucleic acid quadrupled, the unit lattice being made up of eight nucleobases; and
a nanoparticle bound to an end of the nucleic acid quadruplex.

2. The nucleic acid nanostructure of claim 1, wherein the substrate is selected from the group consisting of a metal substrate, a glass substrate, a semiconductor wafer, a quartz substrate, and a plastic substrate.

3. The nucleic acid nanostructure of claim 1, wherein the nucleic acid is selected from the group consisting of DNA, RNA, PNA, LNA, and a hybrid thereof.

4. The nucleic acid nanostructure of claim 1, wherein the nucleic acid quadruplex is composed of four nucleic acid strands which are arranged in a parallel or antiparallel orientation.

5. The nucleic acid nanostructure of claim 1, wherein the nucleic acid quadruplex is composed of four nucleic acid strands which are arranged in parallel with each other in a 5′ to 3′ direction from the substrate.

6. The nucleic acid nanostructure of claim 1, wherein each of the four nucleic acid strands of the nucleic acid quadruplex comprises a guanine-rich sequence.

7. The nucleic acid nanostructure of claim 1, wherein each of the four nucleic acid strands of the nucleic acid quadruplex comprises a sequence selected from the group consisting of sequences as set forth in SEQ ID NOS: 1 through 3.

8. The nucleic acid nanostructure of claim 1, wherein the metal ion is selected from the group consisting of Na⁺, K⁺, Mg²⁺, Ca²⁺, Mn²⁺, Ni²⁺, Cd²⁺, Co²⁺, and Zn²+.

9. The nucleic acid nanostructure of claim 1, wherein the nanoparticle is at least one selected from the group consisting of Au, Ag, ZnS, CdS, CdSe, SiO₂, SnO₂, TiO₂, GaAs, and InP.

10. A method of manufacturing a nucleic acid nanostructure, the method comprising:
introducing a nucleic acid capable of forming a quadruplex onto a substrate;
forming a nucleic acid quadruplex from the introduced nucleic acid; and
binding a nanoparticle to an end of the nucleic acid quadruplex.

11. The method of claim 10, wherein in the introduction of the nucleic acid, a functional group is bound to an end of the nucleic acid capable of forming the quadruplex, and the functional group-containing nucleic acid is immobilized on the substrate.

12. The method of claim 10, wherein in the introduction of the nucleic acid, the nucleic acid capable of forming the quadruplex is in-situ grown on the substrate.

13. The method of claim 10, wherein in the formation of the nucleic acid quadruplex, a metal ion is supplied to the introduced nucleic acid.

14. The method of claim 10, wherein in the binding of the nanoparticle, the nanoparticle is supplied to the nucleic acid quadruplex.
